SEQUENCE LISTING (19 sequences)

SEQ ID NO: 1 - protein, 119 aa, synthetic construct
QVQLVQSGAEVKKPGASVKVSCKATGYTFSNYWIEWVRQAPGQGLEWIGEIFPGSGRINFNEKFKGRVTFTADTSISTTYMELSRLRSDDTAVYYCARTKIYGNSFDYWGQGTLVTVSS

SEQ ID NO: 2 - protein, 107 aa, synthetic construct
DIQMTQSPSSLSASVGDRVTITCKASQDVGTAVVWYQQKPGKAPKLLIYWASSRHEGVPDRFSGSGSGTDFTLTISSLQPEDFADYFCQQYSRYPLTFGQGTKLEIK

SEQ ID NO: 3 - protein, 5 aa, synthetic construct
NYWIE

SEQ ID NO: 4 - protein, 17 aa, synthetic construct
EIFPGSGRINFNEKFKG

SEQ ID NO: 5 - protein, 10 aa, synthetic construct
TKIYGNSFDY

SEQ ID NO: 6 - protein, 11 aa, synthetic construct
KASQDVGTAVV

SEQ ID NO: 7 - protein, 7 aa, synthetic construct
WASSRHE

SEQ ID NO: 8 - protein, 9 aa, synthetic construct
QQYSRYPLT

SEQ ID NO: 9 - protein, 119 aa, synthetic construct
  VARIANT 11: note = X=L or V
  VARIANT 20: note = X=I or V
  VARIANT 37: note = X=I or V
  VARIANT 38: note = X=K or R
  VARIANT 48: note = X=I or M
  VARIANT 68: note = X=A or V
  VARIANT 77: note = X=D or S
  VARIANT 79: note = X=T or A
  VARIANT 82: note = X=Q or E
  VARIANT 85: note = X=S or R
  VARIANT 87: note = X=T or R
  VARIANT 91: note = X=S or T
  VARIANT 114: note = X=T or L
QVQLVQSGAEXKKPGASVKXSCKATGYTFSNYWIEWXXQAPGQGLEWXGEIFPGSGRINFNEKFKGRXTFTADTSIXTXYMXLSXLXSDDXAVYYCARTKIYGNSFDYWGQGTXVTVSS

SEQ ID NO: 10 - protein, 107 aa, synthetic construct
  VARIANT 3: note = X=M or Q
  VARIANT 10: note = X=F or S
  VARIANT 24: note = X=K or R
  VARIANT 34: note = X=V or A
  VARIANT 46: note = X=L or S
  VARIANT 60: note = X=D or S
  VARIANT 63: note = X=T or S
  VARIANT 80: note = X=S or P
  VARIANT 85: note = X=D or T
DIXMTQSPSXLSASVGDRVTITCXASQDVGTAVXWYQQKPGKAPKXLIYWASSRHEGVPXRFXGSGSGTDFTLTISSLQXEDFAXYFCQQYSRYPLTFGQGTKLEIK

SEQ ID NO: 11 - protein, 119 aa, synthetic construct
QVQLVQSGAELKKPGASVKISCKATGYTFSNYWIEWIKQAPGQGLEWIGEIFPGSGRINFNEKFKGRATFTADTSIDTTYMQLSSLTSDDSAVYYCARTKIYGNSFDYWGQGTTVTVSS

SEQ ID NO: 12 - protein, 119 aa, synthetic construct
QVQLVQSGAEVKKPGASVKISCKATGYTFSNYWIEWIKQAPGQGLEWIGEIFPGSGRINFNEKFKGRATFTADTSIDTTYMELSRLRSDDTAVYYCARTKIYGNSFDYWGQGTLVTVSS

SEQ ID NO: 13 - protein, 119 aa, synthetic construct
QVQLVQSGAEVKKPGASVKVSCKATGYTFSNYWIEWIKQAPGQGLEWIGEIFPGSGRINFNEKFKGRVTFTADTSISTTYMELSRLRSDDTAVYYCARTKIYGNSFDYWGQGTLVTVSS

SEQ ID NO: 14 - protein, 119 aa, synthetic construct
QVQLVQSGAEVKKPGASVKVSCKATGYTFSNYWIEWVRQAPGQGLEWMGEIFPGSGRINFNEKFKGRVTFTADTSISTAYMELSRLRSDDTAVYYCARTKIYGNSFDYWGQGTLVTVSS

SEQ ID NO: 15 - protein, 107 aa, synthetic construct
DIMMTQSPSFLSASVGDRVTITCKASQDVGTAVVWYQQKPGKAPKLLIYWASSRHEGVPDRFTGSGSGTDFTLTISSLQSEDFADYFCQQYSRYPLTFGQGTKLEIK

SEQ ID NO: 16 - protein, 107 aa, synthetic construct
DIQMTQSPSSLSASVGDRVTITCRASQDVGTAVVWYQQKPGKAPKLLIYWASSRHEGVPSRFSGSGSGTDFTLTISSLQPEDFATYFCQQYSRYPLTFGQGTKLEIK

SEQ ID NO: 17 - protein, 107 aa, synthetic construct
DIQMTQSPSSLSASVGDRVTITCRASQDVGTAVAWYQQKPGKAPKSLIYWASSRHEGVPSRFSGSGSGTDFTLTISSLQPEDFATYFCQQYSRYPLTFGQGTKLEIK

SEQ ID NO: 18 - protein, 446 aa, synthetic construct
QVQLVQSGAEVKKPGASVKVSCKATGYTFSNYWIEWVRQAPGQGLEWIGEIFPGSGRINFNEKFKGRVTFTADTSISTTYMELSRLRSDDTAVYYCARTKIYGNSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 214 aa, synthetic construct
DIQMTQSPSSLSASVGDRVTITCKASQDVGTAVVWYQQKPGKAPKLLIYWASSRHEGVPDRFSGSGSGTDFTLTISSLQPEDFADYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0014268 A1
(43) Pub. Date: Jan. 15, 2026

(54) DRUG CONJUGATES OF HUMANIZED ANTI PVR ANTIBODIES

(71) Applicant: NECTIN THERAPEUTICS LTD., Jerusalem (IL)

(72) Inventors: Pinchas TSUKERMAN, Jerusalem (IL); Anas ATIEH, Jerusalem (IL); Akram OBIEDAT, Jerusalem (IL); Alon VITENSHTEIN, Jerusalem (IL); Guy CINAMON, Tel Aviv (IL)

(73) Assignee: NECTIN THERAPEUTICS LTD., Jerusalem (IL)

(21) Appl. No.: 18/993,887

(22) PCT Filed: Sep. 4, 2023
(86) PCT No.: PCT/IL2023/050942

§ 371 Date: Jan. 13, 2025

Related U.S. Application Data
(60) Provisional application No. 63/403,750, filed on Sep. 4, 2022.

(51) Int. Cl. A61K 47/68; A61P 35/00; C07K 16/28; G01N 33/574

(52) U.S. Cl. CPC A61K 47/6851; A61K 47/6803; A61K 47/68031; A61K 47/68033; A61P 35/00; C07K 16/2896; G01N 33/57492

(57) ABSTRACT

The present invention provides antibody drug conjugates (ADCs) of humanized anti-PVR (CD155) and use thereof in treating diseases, in particular cancer.

### FIELD OF THE INVENTION

The invention is in the field of immunotherapy and relates to antibody drug conjugates (ADCs) of humanized anti-PVR (CD155) and use thereof in treating diseases, in particular cancer.

### BACKGROUND OF THE INVENTION

The specificity of antibodies for antigens on the surface of target cells and molecules has led to their extensive use as carriers of a variety of diagnostic and therapeutic agents. For example, antibodies conjugated to labels and reporter groups such as fluorophores, radioisotopes and enzymes find use in labelling and imaging applications, while conjugation to cytotoxic agents and chemotherapy drugs allows targeted delivery of such agents to specific tissues or structures, for example particular cell types or growth factors, minimizing the impact on normal, healthy tissue and significantly reducing the side effects associated with chemotherapy treatments. Antibody-drug conjugates (ADC) have extensive potential therapeutic applications in several disease areas, particularly in cancer, and become a novel targeted drug for disease treatment. A typical ADC contains an antibody for targeting, a connector or linker for drug attachment and a high potent payload (e.g., a drug) as effector. Since the approvals of Adcetris in 2011 and Kadcyla in 2013 by US FDA, ADC drug development has widely spread for the treatment of cancer. Over 80 ADCs are currently in clinical development and eleven ADCs (nine containing small-molecule payloads and two with biological toxins) were approved for use by the FDA (Baah et al. 2021 May 15; 26 (10): 2943).

The conjugated drugs may belong to various chemical families. For example, auristatin is a microtubule-destroying drug, derived from marine shell-less mollusk Dolabella auricularia called dolastatins. Various derivatives of auristatin have been synthesized, such as monomethyl auristatin E (MMAE) and monomethyl auristatin F (MMAF). MMAE and MMAF were developed by Seattle Genetics and used as payloads for ADCs. MMAF and MMAE have their advantages and disadvantages. MMAE is more membrane-permeable and has a lower IC50 than MMAF. However, MMAF is more hydrophilic and has a lower aggregation tendency to show lower systemic toxicity than MMAE (park et al. Molecules 2019, 24, 2754). Ravtansine (DM4) is another example of toxin that may be use in ADCs. Ravtansine is a maytansinoid, a chemical derivative of maytansine, which has the ability to disrupt microtubule function. Another maytansinoid, emtansine (DM1), binds at the ends of microtubules and thereby suppress their dynamic instability.

Poliovirus receptor (PVR), also termed CD155, is a transmembrane glycoprotein involved in mediating cell adhesion to extracellular matrix molecules. It was previously described as a tumor antigen and as a potential target for therapeutic intervention as its expression is up-regulated in neuroectodermal cancers, including glioblastoma multiforme, medulloblastoma, and colorectal carcinoma (Solecki et al., J. Biol. Chem. 2002, 277:25697-700), as well as in pancreatic cancer (Nishiwada et al., Anticancer Res. 2015, 35 (4): 2287-97). PVR is also known to enhance the serum-induced activation of the Ras-Raf-MEK-ERK signaling, up-regulating cyclins D2 and E, and down-regulated p27Kip1, eventually shortening the period of the G0/G1 phase of the cell cycle (Kakunaga 2004, J. Biological Chemistry, 279, 36419-36425. For that reason, blocking of PVR on tumor cells is anticipated to reduce their viability. PVR has also a critical role in angiogenesis and is suggested to regulate the VEGF-induced angiogenesis by controlling the interaction of Vascular endothelial growth factor receptor 2 (VEGFR2) with integrin a (v) β (3), and the VEGFR2-mediated Rap1-Akt signaling pathway (Kinugasa et al., 2012. Circ Res. 2012, 110 (5), 716-26). Additionally, PVR is complexing with IGFIR and participating in tyrosine-protein kinase Met (cMet) signaling and blocking the complex formation reduced cell viability and angiogenesis (Lee et al., Scientific Reports 2014, 20, 4, 7139).

In recent years it became evident that PVR is a critical immune check point ligand (Brilc P. K. et al 2019 Cell Mol Immunology). PVR expression is upregulated in both malignant cells and tumor-infiltrating myeloid cells in humans and mice. PVR−/− mice display reduced tumor growth and metastasis via DNAM-1 (CD226) upregulation and enhanced effector function of CD8+T and NK cells, respectively. Blockade of Programmed cell death protein 1 (PD-1) or both PD-1 and cytotoxic T-lymphocyte-associated protein 4 (CTLA4) is more effective in settings in which PVR was limiting, suggesting the clinical potential of combinatory therapy using PD-1/PD-L1 and PVR blockade. Moreover, in clinical settings, the expression of PD-L1 and PVR is independently regulated, which allowed stratification of patients who were treated with anti-PD-1 antibody into 4 groups according to the expression levels of PD-L1 and PVR. High PVR expression in PD-L1-low-expressing patients enriched non-responders. This was further validated using a genetically engineered cancer model. These findings bolster the significance of PVR as a critical immune check point in tumor immune-therapy (Lee B. R et al., JCI. Insight 2020). PVR involvement in metastasis was demonstrated by injecting cancer cells to the tail of mice and measuring metastasis to the lungs. It has been shown that the upregulated PVR in cancer cells trans-interacts with its counter-receptor in platelets, and that this trans-interaction enhances the metastasis of the cancer cells to the lungs (Morimoto et al., Oncogene (2008) 27, 264-273).

WO 2017/149538, to some of the inventors of the present invention, discloses murine antibodies and fragments thereof which bind to PVR as well as encoding polynucleotide sequences and hybridoma cells producing these antibodies.

U.S. Patent Application Publication No. 2007/0041985 discloses molecules that specifically bind to at least one intra- or extracellular domain of the PVR, wherein the molecules have the ability to modulate a receptor mediated adhesion, trafficking and/or invasion behavior of a cell expressing the PVR or any derivative thereof.

U.S. Patent Application Publication No. 2009/0215175 provides molecules (e.g., small chemical compounds, oligonucleotides, polypeptides, antibodies, and antibody fragments) which modulate the PVR functions necessary for adhesion, trafficking, invasion and/or metastatic potential of cells. The molecules can be used for the treatment of cells having a metastatic potential, metastasis and cancer.

WO 2021/070181 to some of the inventors of the present invention discloses humanized anti-PVR antibodies, capable of restoring immune activity and upregulating the surface expression of DNAM1 (CD226) on CD8 and NK cells. The humanized antibodies are particular useful for treatment of PVR-expressing tumors. One of the disclosed antibodies is NB1088 (denoted herein NTX1088).

U.S. Patent Application Publication No. 2022/0056146 discloses a humanized antibody or antigen binding fragment that binds to the poliovirus receptor (PVR) can be utilized in the preparation of antibody drug conjugates (ADCs) to target nucleic acids, peptides and proteins, drugs and radiopharmaceuticals to cancer cells.

WO 2019/102456 discloses immunotoxins for treating cancer and describes anti-PVR antibodies and immunotoxins thereof for treating glioblastoma.

While several ADCs are approved for clinical use, showing robust efficacy in several indications, they are also associated with significant toxicity which may limit their use. Such toxicity may be caused by the antibody recognizing the target on healthy tissues, or by the drug being released to the circulation. Thus, there is an unmet need to provide novel ADCs that are tumor specific, that are safe and potent and can be used as new cancer therapies.

### SUMMARY OF THE INVENTION

The present invention provides antibody drug conjugates (ADCs) that comprise an antibody specific to human PVR (CD155), or antigen binding portion thereof, and a cytotoxin. According to some embodiments, the antibody is a humanized antibody. The ADCs provided herein, in some embodiments, are useful in treating cancers that express PVR, in particular hard-to-treat tumors.

It is now disclosed that ADCs comprising certain payloads that are linked to a specific humanized anti-PVR antibody, named NTX1088, are highly efficient in targeting therapeutic and diagnostic agents to cancers cells, including resistant types of cancer. The ADCs were found to be highly specific with minimal effect on non-tumor cells (PVR-negative target cells or non-dividing PVR positive, normal cells). The ADCs described herein were found to be more efficient and safer than previously published anti-PVR ADCs and may be useful in treating and diagnosing a variety of cancers.

The selection of a proper linker and toxin to achieve maximal therapeutic efficacy without excessive toxicity, is nonobvious and requires undue experimentation. The combination of the antibody-linker-toxin must ensure release of the toxin in the target cells, and effective killing activity of these cell while minimizing damage to normal cells. Not any high specific antibody will be suitable for use in an ADC as it has to enable simultaneously rapid internalization, which is crucial for both efficacy and safety, since it reduces the opportunity of the ADC for off-target release. Moreover, ADC candidates must be designed, synthesized and tested in vitro and more so, in vivo, in order to judge their efficacy and toxicity. It is now disclosed that the specific humanized anti-PVR antibodies described herein, in particular NTX1088, are highly efficient and suitable for use in ADCs.

The ADCs described herein were found to be efficient against highly resistant cancers such as glioblastoma, as demonstrated in the common GBM model cells U87. These results are in sharp contrast to previously attempts to use other anti-PVR ADCs against highly resistant tumors.

According to one aspect, the present invention provides an antibody-drug conjugate (ADC) comprising a humanized anti-PVR antibody or antigen binding portion thereof, conjugated to an active or a detectable moiety (payload), the antibody or antigen binding portion thereof comprises a heavy chain and a light chain, wherein the heavy chain comprises a variable region having an amino acid sequence at least about 90% identical to SEQ ID NO: 1, and wherein the light chain comprises a variable region having an amino acid sequence at least about 90% identical to SEQ ID NO: 2.

According to some embodiments, the humanized antibody comprises: a heavy-chain variable region amino-acid sequence comprising the CDR sequences set forth in SEQ ID NO: 3 (NYWIE), SEQ ID NO: 4 (EIFPGSGRINFNEKFKG) and SEQ ID NO: 5 (TKIYGNSFDY), and a light-chain variable region amino-acid sequence comprising the CDR sequences set forth in SEQ ID NO: 6 (KASQDVGTAVV), SEQ ID NO: 7 (WASSRHE) and SEQ ID NO: 8 (QQYSRYPLT).

According to some embodiments, the humanized antibody comprises a heavy chain comprising a variable region having an amino acid sequence at least about 91%, 92%, 93%, 94%, 95%, 96%, 97%, 98%, 99% or 99% identical to SEQ ID NO: 1. Each possibility represents a separate embodiment of the invention. According to some embodiments, the humanized antibody comprises a heavy chain comprising a variable region having an amino acid sequence at least about 95% identical to SEQ ID NO: 1. According to some embodiments, the humanized antibody comprises a heavy chain variable region having an amino acid sequence set forth in SEQ ID NO: 1.

According to some embodiments, the humanized antibody comprises a light chain comprising a variable region having an amino acid sequence at least about 91%, 92%, 93%, 94%, 95%, 96%, 97%, 98%, 99% or 99% identical to SEQ ID NO: 2. Each possibility represents a separate embodiment of the invention. According to some embodiments, the humanized antibody comprises a light chain comprising a variable region having an amino acid sequence at least about 95% identical to SEQ ID NO: 2. According to some embodiments, the humanized antibody comprises a light chain variable region having an amino acid sequence set forth in SEQ ID NO: 2.

According to some embodiments, the humanized antibody comprises a heavy chain variable region having an amino acid sequence set forth in SEQ ID NO: 1 and a light chain variable region having an amino acid sequence set forth in SEQ ID NO: 2 (denoted herein NTX1088).

According to some embodiments, the humanized antibody is an IgG antibody. According to some embodiments, the humanized antibody has a heavy chain constant region selected from IgG4 and IgG1. In certain embodiments, the humanized antibody or antigen binding portion thereof is an IgG4 subclass. In certain embodiments, the humanized antibody or antigen binding portion thereof is an IgG1 subclass.

According to some embodiments, the humanized antibody or fragment thereof comprises a human IgG4 constant region having S228P (also named S241P) substitution in the hinge region.

According to some embodiments, the humanized antibody comprises a heavy chain having an amino acid sequence set forth in SEQ ID NO: 18 and a light chain having an amino acid sequence set forth in SEQ ID NO: 19.

According to some embodiments, the humanized antibody or antigen binding portion thereof is a full antibody, a Fab, a F(ab)₂, a single-domain antibody, or a single chain variable fragment (scFv).

According to some embodiments, the active moiety (payload) is a toxin.

According to other embodiments, the payload is a detectable moiety such as a radioactive or a fluorescent moiety.

Any chemical or biological entity that capable of killing or inhibiting the growth of tumor cells in vivo may be used, as a toxin, with the ADCs of the present invention. According to some embodiments, the toxin is selected from the group consisting of microtubule inhibitor, DNA synthesis inhibitor, topoisomerase inhibitor and RNA polymerase inhibitor.

According to certain embodiments, the toxin is a microtubule-destroying drug. According to certain exemplary embodiments, the toxin is auristatin or a derivative thereof. According to certain embodiments, the auristatin derivative is monomethyl auristatin E (MMAE) or monomethyl auristatin F (MMAF).

According to some embodiments, the toxin is saporin.

According to some embodiments, the toxin is a maytansine derivative. According to certain embodiments, the maytansine derivative is DM4 or DM1.

According to some embodiments, the toxin is a quinoline alkaloid. According to certain embodiments, the quinoline alkaloid is SN-38.

According to some embodiments, the toxin is selected from the group consisting of DM4, MMAE and SN-38. According to certain embodiments, the toxin is DM4 or MMAE.

According to some embodiments, the toxin is a topoisomerase I inhibitor. According to some embodiments, the toxin is a derivative of camptothecin. According to certain exemplary embodiments, the toxin is Exatecan.

According to certain exemplary embodiments, the ADC comprises a toxin selected from the group consisting of Saporin, MMAE, DM1, DM4, SN-38, and Exatecan. Each possibility represents a separate embodiment of the invention.

According to some embodiments, the toxin is linked directly to the antibody. According to other embodiments, the antibody and the toxin are linked through a linker. According to some embodiments, the toxin is covalently linked to the humanized antibody directly or through a linker.

According to some embodiments, the linker is cleavable. According to other embodiments, the linker is not cleavable. According to some embodiments, the cleavable linker is selected from the group consisting of an enzymatic cleavable linker, a pH-sensitive linker and a reducible linker. According to some embodiments, the linker is an enzymatic cleavable linker. According to certain embodiments, the linker is a pH-sensitive linker. According to some embodiments, the linker is a reducible linker.

According to some embodiments, the linker is selected from the group consisting of Maleimidocaproyl (MC), Maleimidocaproyl-Valine-Citrulline-p-amino-benzyloxycarbonyl (MC-VC-PAB), Maleimidomethyl cyclohexane-1-carboxylate (SMCC), N-succinimidyl-4-(2-pyridyldithio) butanoate (sulfo-SPDB) and Lys-PAB-CO (Lysine-p-aminobenzyl-C═O).

According to yet other embodiments, the linker comprises a stretch of 1-30 amino acids residues. According to some embodiments, the linker comprises two cysteine residues that are used for cysteine conjugation. According to certain exemplary embodiments, the linker comprises Valine (Val) and Alanine (Ala) residues. According to some embodiments, the linker is a Valine-Alanine (VA) linker, namely comprising a stretch of 2-20 amino acids consisting of Ala and Val residues. According the certain embodiments, the linker consists of the amino acids Valine-Alanine.

According to some embodiments, the ADC comprises the antibody NTX1088.

According to some embodiments, the ADC comprises the antibody NTX1088 and a toxin selected from the group consisting of MMAE, MMAF, DM1, DM4, SN-38, and Exatecan.

According to some embodiments, the ADC comprises the antibody NTX1088, the toxin MMAE and the linker MC-VC-PAB (denoted herein NTX1088-MMAE). According to some embodiments, the ADC comprises the toxin MMAF and the linker MC (denoted herein NTX1088-MMAF). According to some embodiments, the conjugate comprises the toxin DM1 and the linker SMCC (denoted herein NTX1088-DM1). According to some embodiments, the conjugate comprises the toxin DM4 and the linker SPDB (denoted herein NTX1088-DM4). According to some embodiments, the conjugate comprises the toxin SN38 and the linker Lys-PAB-CO (denoted herein NTX1088-SN38). According to some embodiments, the conjugate comprises the toxin Exatecan and a Valine-Alanine linker (denoted herein NTX1088-Exatecan).

According to some embodiments, the ADC comprises an anti-PVR antibody that competes with an antibody described herein to specifically bind to the PVR molecule. According to certain embodiments, the ADC comprises an anti-PVR antibody that competes with an antibody comprising a heavy-chain variable region amino-acid sequence comprising the CDR sequences set forth in SEQ ID NO: 3 (NYWIE), SEQ ID NO: 4 (EIFPGSGRINFNEKFKG) and SEQ ID NO: 5 (TKIYGNSFDY), and a light-chain variable region amino-acid sequence comprising the CDR sequences set forth in SEQ ID NO: 6 (KASQDVGTAVV), SEQ ID NO: 7 (WASSRHE) and SEQ ID NO: 8 (QQYSRYPLT), to specifically bind to the PVR molecule.

The present invention provides, according to another aspect, a pharmaceutical composition comprising the conjugate described herein and a pharmaceutically acceptable excipient, carrier, or diluent.

Any administration mode may be used to deliver the compositions of the present invention to a subject in need thereof, including parenteral and enteral administration modes.

According to some embodiments, the pharmaceutical composition is formulated for injection or infusion. According to some embodiments, the pharmaceutical composition is formulated for intravenous (IV) administration. In certain embodiments, the pharmaceutical composition is formulated for intratumoral (IT) administration.

According to some embodiments, the conjugate or the pharmaceutical composition is for use in treating a cancer in an individual.

According to some embodiments, the cancer is a resistant type of cancer. According to some embodiments, the cancer is characterized by expression of PVR. According to some embodiments, the cancer is characterized by high expression of PVR. According to some embodiments, the cancer is characterized by overexpression of PVR.

In certain embodiments, the cancer comprises a solid tumor. In certain embodiments, the cancer is selected from the group consisting of liver cancer, lung cancer, colon cancer, glioblastoma, adrenal cancer, uterine cancer, testis cancer, head and neck cancer, pancreatic cancer, and breast cancer. Each possibility represents a separate embodiment of the invention. According to some embodiments, the cancer is glioblastoma. According to some embodiments, the cancer is colorectal adenocarcinoma. According to certain embodiments, the cancer is non-small cell lung carcinoma (NSCLC).

According to additional embodiments, the cancer is selected from the group consisting of Cholangiocarcinoma, Colon adenocarcinoma, Esophageal carcinoma, Head and Neck squamous cell carcinoma. Kidney renal papillary cell carcinoma, Liver hepatocellular carcinoma, Lung adenocarcinoma, Pancreatic adenocarcinoma, Prostate adenocarcinoma, Skin Cutaneous Melanoma, Stomach adenocarcinoma, Testicular Germ Cell Tumors, Uterine Corpus Endometrial Carcinoma, Bladder Urothelial Carcinoma, Pheochromocytoma and Paraganglioma, Lung squamous cell carcinoma, Mesothelioma, Cervical squamous cell carcinoma, endocervical adenocarcinoma, Kidney Chromophobe Gastric carcinoma, Gastroesophageal junction carcinoma. Small cell lung carcinoma, Adrenal carcinoma, Gallbladder carcinoma, Small intestine carcinoma, and Anal carcinoma. Each possibility represents a separate embodiment of the invention.

According to some embodiments, the cancer is selected from the group consisting of lung cancer, cervical cancer, urothelial cancer, pancreatic cancer, ovarian cancer, colorectal cancer, hepatocellular cancer, esophageal cancer, and a brain tumor.

According to some embodiments, the cancer is a hematological cancer.

According to some embodiments, the hematological cancer is selected from leukemia including acute myeloid leukemia (AML), chronic myeloid leukemia (CML), acute lymphocytic leukemia (ALL), and chronic lymphocytic leukemia (CLL); lymphoma, including Hodgkin disease, and non-Hodgkin lymphoma; and multiple myeloma.

According to some embodiments, the cancer is a resistant and/or hard-to-treat cancer.

According to some embodiments, the individual is human.

According to some embodiments, the use further comprises an agent that downregulates the activity or expression of an immune co-inhibitory receptor.

According to some embodiments, the immune co-inhibitory receptor is selected from the group consisting of PD-1, PD-L1, TIGIT, CTLA-4, LAG3, TIM3, BTLA, VISTA, B7H4, CD96, BY55 (CD 160), LAIR1, SIGLEC10, CD112R, CD112, ILT-4 and 2B4. Each possibility represents a separate embodiment of the invention.

According to some embodiments of the invention, the use further comprises the use in a combination with an additional ADC.

According to an additional aspect, the present invention provides an antibody-drug conjugate (ADC) comprising a humanized anti-PVR antibody conjugated to a toxin, for use in treating a resistant or hard-to-treat cancer, wherein the antibody comprises: a heavy-chain variable region amino-acid sequence comprising the CDR sequences set forth in SEQ ID NO: 3 (NYWIE), SEQ ID NO: 4 (EIFPGSGRINFNEKFKG) and SEQ ID NO: 5 (TKIYGNSFDY), and a light-chain variable region amino-acid sequence comprising the CDR sequences set forth in SEQ ID NO: 6 (KASQDVGTAVV), SEQ ID NO: 7 (WASSRHE) and SEQ ID NO: 8 (QQYSRYPLT).

According to some embodiments, the antibody comprises a heavy chain comprising a variable region having a sequence set forth in SEQ ID NO: 1, and a light chain comprising a variable region having a sequence set forth in SEQ ID NO: 2.

According to an additional aspect, the present invention provides an antibody-drug conjugate (ADC) comprising a humanized anti-PVR antibody conjugated to a toxin, for use in treating a resistant or hard-to-treat cancer, wherein the antibody is NTX1088.

The present invention provides, according to another aspect, a method of treating a cancer in an individual in need of such treatment, the method comprises administering to the individual a therapeutically effective amount of the conjugate or the pharmaceutical composition described herein. In certain embodiments, the cancer is a solid tumor. According to additional embodiments, the cancer is a non-solid tumor. In certain embodiments, the cancer is selected from the group consisting of glioblastoma, pancreatic cancer, breast cancer, bladder cancer, kidney cancer, head and neck cancer, ovarian cancer, colon cancer, cervical cancer, prostate cancer, and lung cancer. In certain embodiments, the method of treating cancer involves preventing or reducing formation, growth or spread of metastases in a subject.

According to some embodiments, the cancer is a resistant or hard-to-treat cancer.

According to some embodiments, the cancer is resistant to chemotherapy or radiation.

According to some embodiments, the individual has become resistant to prior immunotherapy, chemotherapy or radiation treatment.

According to other embodiments, the cancer is a hematological cancer. According to some embodiments, the hematological cancer is selected from leukemia including acute myeloid leukemia (AML), chronic myeloid leukemia (CML), acute lymphocytic leukemia (ALL), and chronic lymphocytic leukemia (CLL); lymphoma, including Hodgkin disease, and non-Hodgkin lymphoma; and multiple myeloma.

According to some embodiments, the individual is human.

According to some embodiments, the method of treating cancer comprises administering or performing at least one additional anti-cancer therapy. According to certain embodiments, the additional anticancer therapy is surgery, chemotherapy, radiotherapy, or immunotherapy.

According to some embodiments, the method of treating cancer comprises administration of the conjugate described herein and an additional anti-cancer agent. According to some embodiments, the additional anti-cancer agent is selected from the group consisting of: immune-modulator, activated lymphocyte cell, kinase inhibitor and chemotherapeutic agent.

According to some embodiments, the additional immune-modulator is an antibody against an immune checkpoint molecule. According to some embodiments, the additional immune modulator is an antibody against an immune checkpoint molecule selected from the group consisting of human programmed cell death protein 1 (PD-1). PD-L1 and PD-L2, carcinoembryonic antigen-related cell adhesion molecule 1 (CEACAM1), lymphocyte activation gene 3 (LAG3), CD137, OX40 (also referred to as CD134), killer cell immunoglobulin-like receptors (KIR), TIGIT, Nectin-2, CTLA-4, NKG2A. GITR, and any other checkpoint molecule or a combination thereof. Each possibility represents a separate embodiment of the invention. According to certain embodiments, the additional immune modulator is an antibody against PD-1. According to some embodiments, the additional immune modulator is an antibody against CTLA-4.

According to some embodiments, the method of treating cancer involves preventing or reducing formation, growth or spread of metastases in a subject.

Also described herein is a method of making composition for treating a cancer in an individual afflicted with cancer comprising admixing the conjugate described herein and a pharmaceutically acceptable excipient, carrier, or diluent. In certain embodiments, the cancer comprises a solid tumor. In certain embodiments, the cancer is selected from the group consisting of glioblastoma, pancreatic cancer, breast cancer, bladder cancer, kidney cancer, head and neck cancer, ovarian cancer, colon cancer, cervical cancer, prostate cancer, and lung cancer.

According to another aspect, the present invention provides an antibody-drug conjugate (ADC) comprising a humanized anti-PVR antibody, or antigen binding portion thereof, conjugated to a radioactive moiety, or labeling tag, the antibody or antigen binding portion thereof comprises a heavy chain and a light chain, wherein the heavy chain comprises a variable region having an amino acid sequence at least about 90% identical to SEQ ID NO: 1, and wherein the light chain comprises a variable region having an amino acid sequence at least about 90% identical to SEQ ID NO: 2.

The humanized antibody is as described hereinabove.

According to another aspect, the present invention provides a method of delivering an antibody-drug conjugate (ADC) to a cell comprising contacting the cell with the ADC, wherein the ADC comprises a humanized anti-PVR antibody, or antigen binding portion thereof, conjugated to a toxin, the antibody or antigen binding portion thereof comprises a heavy chain and a light chain, wherein the heavy chain comprises a variable region having an amino acid sequence at least about 90% identical to SEQ ID NO: 1, and wherein the light chain comprises a variable region having an amino acid sequence at least about 90% identical to SEQ ID NO: 2.

Any ADC described above may be used in the method of the present invention. According to certain embodiments, the ADC comprises humanized antibody comprising: a heavy-chain variable region amino-acid sequence comprising the CDR sequences set forth in SEQ ID NO: 3 (NYWIE), SEQ ID NO: 4 (EIFPGSGRINFNEKFKG) and SEQ ID NO: 5 (TKIYGNSFDY), and a light-chain variable region amino-acid sequence comprising the CDR sequences set forth in SEQ ID NO: 6 (KASQDVGTAVV), SEQ ID NO: 7 (WASSRHE) and SEQ ID NO: 8 (QQYSRYPLT).

According to some embodiments, the cells are tumor cells.

According to some embodiments, the method comprises administering the ADC to cells of a subject. According to certain embodiments, the subject is a human subject.

According to some embodiments, the cells are of a cancer expressing or overexpressing PVR. According certain embodiments, the cells are of a cancer selected from the group consisting of a melanoma, a breast cancer, an ovarian cancer, a pancreatic cancer, a colorectal cancer, a colon cancer, a cervical cancer, a kidney cancer, a lung cancer, a thyroid cancer, a prostate cancer, a brain cancer, a renal cancer, a throat cancer, a laryngeal carcinoma, a bladder cancer, a hepatic cancer, a fibrosarcoma, an endometrial cells cancer, a glioblastoma, sarcoma, a myeloid, a leukemia and a lymphoma.

According to some embodiments, the method further comprises administering to said subject an additional immuno-modulator, activated lymphocyte cells, kinase inhibitor, chemotherapeutic agent or any other anti-cancer agent.

According to another aspect, the present invention provides a method of delivering an ADC as described herein to a cell of a subject, said method comprising administering the conjugate to the subject.

The present invention further provides, according to an aspect, a method of diagnosing or prognosing cancer in a subject, the method comprises determining the expression level of PVR in a biological sample of said subject using at least one antibody conjugate as described herein.

The diagnosed cancer is any of the cancer types described hereinabove, in particular a cancer that expresses PVR.

The present invention further provides, according to another aspect, a method of determining or quantifying the expression of PVR, the method comprising contacting a biological sample with an antibody conjugate as described herein, and measuring the level of complex formation.

According to some embodiments, the method for detecting or quantifying the expression of PVR comprises the steps of:
i. incubating a sample with the antibody conjugate described herein;
ii. detecting the bound PVR using said conjugate.

According to some embodiments, the method further comprises the steps of:
iii. comparing the amount of (ii) to a standard curve obtained from a reference sample containing a known amount of PVR; and
iv. calculating the amount of the PVR in the sample from the standard curve.

According to some particular embodiments, the sample is a body fluid or solid tissue. In some embodiments, the method is performed in-vitro or ex-vivo.

A kit for measuring the expression of PVR in biological sample is also provided comprising at least one conjugate as described herein and means for measuring PVR expression. In some embodiment, the kit further comprises instruction material directing the use of the kit.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C show the correlation between PVR expression levels (high or low mRNA levels, as indicated by the dark and light curves, respectively) to survival probability over time. The correlation was significant (p<0.001) for cervical cancer (FIG. 1A), urothelial cancer (FIG. 1B) and lung cancer (FIG. 1C). Data sets were obtained from the TCGA and analyzed by Protein Atlas.

FIG. 2 shows the broad and robust expression of human PVR across biopsies of most prevalent solid cancer types, as measured by immunohistochemistry and evaluated by H-score.

FIGS. 3A-3B demonstrate enhanced potency of the humanized anti-PVR NTX1088 based ADCs in inducing killing of PVR+ EGFR+ glioblastoma multiforme (GBM) cell lines U251 (FIG. 3A) and U87 (FIG. 3B). Biotinylated NTX1088 (black bars) or Erbitux (anti-EGFR mAb, grey bars) were the drivers for generating ADCs that are based on the toxin Saporin (ZAP).

FIG. 4 depicts the killing induced by NTX1088-ADC on cells that are positive and negative for PVR expression. ADC was based on Saporin (ZAP) as in FIG. 3. The A549 (lung adenocarcinoma) and MDA-MB-231 (Triple negative breast cancer, TNBC) target cells were confirmed to express PVR. Jeg-3 (Choriocarcinoma) cells which do not express PVR, were used to evaluate the specificity of the ADC.

FIGS. 5A-5D depict that NTX1088-ADCs are able to robustly and specifically induce killing of tumor cells in-vitro. TNBC target cells MDA-MB-231 (FIG. 5A) and MDA-MB-468 (FIG. 5B), colorectal cancer cells RKO (FIG. 5C), and CHO cells that are human PVR null (FIG. 5D) were incubated with the indicated NTX1088-based ADCs.

FIGS. 6A-6B depict that NTX1088-based ADCs are able to induce robust killing of tumor cells representing hard-to-treat cancer types. SKOV-3 cells, an ovarian cancer model, and U87 cells, a GBM model (FIG. 6A and FIG. 6B, respectively), were incubated with NTX1088-ADCs, in concentrations ranging from 12-0.01 μg/ml. Here, the most potent versions of the ADCs, as depicted in FIG. 5 were tested.

FIGS. 7A-7B depict in-vivo efficacy of the selected NTX1088-based ADCs against the aggressive GBM model U87. Nude female mice were implanted subcutaneously (SC) with 5×10⁶ U87 cells. After the tumors reached an average size of ˜160 mm³, a treatment with leading NTX1088-based ADCs has started. Doses of 5 mg/kg were given as indicated by the arrows in FIG. 7A. The effect of the MMAE-based ADC is shown in FIG. 7B for individual mice (without including the other treatments).

FIG. 8 shows representative image of histology micrograph from a liver of female PVRTg21 mouse. The sample exhibits robust membranal expression of human PVR across the entire tissue, supporting the validity of this strain for assessment of the ADC toxicity.

FIG. 9 depicts in-vivo efficacy of the NTX1088-based ADCs against the non-small cell lung carcinoma (NSCLC) model of H322M cells. Nude female mice were implanted subcutaneously (SC) with 5×10⁶ H322M cells. After the tumors exceeded 130 mm³, a treatment with leading NTX1088-based ADCs has started. Doses of 5 mg/kg for the MMAE conjugated NTX1088 (black filled squares) or 10 mg/kg of Exatecan conjugated NTX1088 (vehicle, grey filled circles) were given i.v. as indicated by the arrows. PBS was given as control (vehicle, grey filled circles).

### DETAILED DESCRIPTION

The present invention provides antibody-drug conjugates, or ADCs, comprising the humanized anti-PVR antibodies described herein, which are useful in treating cancer. Advantageously, the ADCs described herein comprise antibodies that are almost fully humanized, thus avoiding the risk of adverse immune response towards the antibodies and are therefore likely to be safe for use in humans. Furthermore, the ADCs described herein are highly potent and suitable for use in treating high resistant cancer.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments. As used herein the term “about” refers to an amount that is near the stated amount by 10% or less.

According to one aspect, the present invention comprises an antibody-drug conjugate (ADC) comprising a humanized anti-PVR antibody, or antigen binding portion thereof, conjugated to a toxin, the antibody or antigen binding portion thereof comprises a heavy chain and a light chain, wherein the heavy chain comprises a variable region having an amino acid sequence at least about 90% identical to SEQ ID NO: 1, and wherein the light chain comprises a variable region having an amino acid sequence at least about 90% identical to SEQ ID NO: 2.

According to some embodiments, the humanized antibody is NTX1088 that comprises heavy chain variable region having an amino acid sequence of SEQ ID NO: 1, and light chain variable region having an amino acid sequence of SEQ ID NO: 2.

According to another aspect, the present invention comprises an antibody-drug conjugate (ADC) comprising a humanized anti-PVR antibody, or antigen binding portion thereof, conjugated to a toxin selected from the group consisting of SN-38, DM1, DM4, MMAE, and MMAF, the humanized antibody or antigen binding portion thereof comprising: (1) heavy-chain variable region amino-acid sequence comprising the CDR sequences set forth in SEQ ID NO: 3 (NYWIE), SEQ ID NO: 4 (EIFPGSGRINFNEKFKG) and SEQ ID NO: 5 (TKIYGNSFDY), and (2) light-chain variable region amino-acid sequence comprising the CDR sequences set forth in SEQ ID NO: 6 (KASQDVGTAVV), SEQ ID NO: 7 (WASSRHE) and SEQ ID NO: 8 (QQYSRYPLT).

According to some embodiments, the humanized antibody or antigen binding fragment thereof comprises a heavy chain variable region comprising the amino acid sequence QVQLVQSGAE (L/V) KKPGASVK (I/V) SCKATGYTFSNYWIEW (I/V) (K/R) QAPGQGLE W (I/M) GEIFPGSGRINFNEKFKGR (A/V) TFTADTSI (D/S) T (T/A) YM (Q/E) LS (S/R) L (T/R) SDD (S/T) AVYYCARTKIYGNSFDYWGQGT (T/L) VTVSS (SEQ ID NO: 9); and a light chain variable region comprising the amino acid sequence

(SEQ ID NO: 10)
DI(M/Q)MTQSPS(F/S)LSASVGDRVTITC(K/R)ASQDVGTAV(V/A)
WYQQKPGKAPK(L/S)LIYWASSRHEGVP(D/S)RF(T/S)GSGSGTDF
TLTISSLQ(S/P)EDFA(D/T)YFCQQYSRYPLTFGQGT KLEIK.

According to some embodiments, the antibody or a fragment thereof comprises a heavy chain and a light chain, wherein the heavy chain comprises a variable region having an amino acid sequence at least about 95% identical to a sequence selected from the group consisting of SEQ ID NO: 11, SEQ ID NO: 12, SEQ ID NO: 13, and SEQ ID NO: 14; and wherein the light chain comprises a variable region having an amino acid sequence at least about 90% identical to a sequence selected from the group consisting of SEQ ID NO: 15, SEQ ID NO: 16 and SEQ ID NO: 17. Sequences Nos: 11-17 are humanized variants of NTX1088 that were selected based on improved producibility and that they can be assembled into complete V region sequences that were devoid of significant T cell epitopes as described in WO2021070181. The variants include Five heavy chain (VH1 to VH5) and 4 light chains (containing the N56E substitution) (Vκ1 to VκK4).

According to some embodiments, the antibody or a fragment thereof comprises a heavy chain and a light chain, wherein the heavy chain comprises a variable region having an amino sequence selected from the group consisting of SEQ ID NO: 11. SEQ ID NO: 12, SEQ ID NO: 13, and SEQ ID NO: 14; and wherein the light chain comprises a variable region having an amino acid sequence selected from the group consisting of SEQ ID NO: 15, SEQ ID NO: 16 and SEQ ID NO: 17.

According to some embodiments, the humanized antibody comprises a combination of a heavy chain variable region and a light chain variable region, wherein the combination is selected from the group consisting of:
i. a heavy chain variable region sequence set forth in SEQ ID NO: 1 and a light chain variable region sequence set forth in SEQ ID NO: 2;
ii. a heavy chain variable region sequence set forth in SEQ ID NO: 12 and a light chain variable region sequence set forth in SEQ ID NO: 16;
iii. a heavy chain variable region sequence set forth in SEQ ID NO: 13 and a light chain variable region sequence set forth in SEQ ID NO: 2;
iv. a heavy chain variable region sequence set forth in SEQ ID NO: 13 and a light chain variable region sequence set forth in SEQ ID NO: 16;
v. a heavy chain variable region sequence set forth in SEQ ID NO: 12 and a light chain variable region sequence set forth in SEQ ID NO: 2;
vi. a heavy chain variable region sequence set forth in SEQ ID NO: 1 and a light chain variable region sequence set forth in SEQ ID NO: 16;
vii. a heavy chain variable region sequence set forth in SEQ ID NO: 14 and a light chain sequence set forth in SEQ ID NO: 2; and
viii. a heavy chain variable region sequence set forth in SEQ ID NO: 14 and a light chain variable region sequence set forth in SEQ ID NO: 16.

According to some embodiments, the heavy chain variable region of the humanized monoclonal antibody comprises an amino acid sequence identical to that set forth in SEQ ID NO: 1, and the light chain variable region comprises an amino acid sequence identical to that set forth in SEQ ID NO: 2.

The conjugate of the invention comprises a humanized antibodies as described herein. The antibodies include monoclonal antibodies, polyclonal antibodies, multispecific antibodies (for example, bispecific antibodies and polyreactive antibodies), and antibody fragments. Thus, an antibody includes, but is not limited to, full-length, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The antibody can comprise a human IgG1 constant region. The antibody can comprise a human IgG4 constant region.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments. Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

There are several methods known in the art for determining the CDR sequences of a given antibody molecule, but there is no standard unequivocal method. Determination of CDR sequences from antibody heavy and light chain variable regions can be made according to any method known in the art, including but not limited to the methods known as KABAT, Chothia and IMGT. A selected set of CDRs may include sequences identified by more than one method, namely, some CDR sequences may be determined using KABAT and some using IMGT, for example. According to some embodiments, the CDR sequences of the mAb variable regions are determined using the IMGT method. For example, CDR determination is made according to the Kabat (Wu T. T and Kabat E. A., J Exp Med, 1970; 132:211-50) and IMGT (Lefranc M-P, et al., Dev Comp Immunol, 2003, 27:55-77).

When the term “CDR having a sequence”, or a similar term is used, it includes options wherein the CDR comprises the specified sequences and also options wherein the CDR consists of the specified sequence.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv or sFv); and multispecific antibodies formed from antibody fragments. In particular embodiments, the antibodies are single-chain antibody fragments comprising a variable heavy chain region and/or a variable light chain region, such as scFvs.

A “humanized” antibody is an antibody in which all or substantially all CDR amino acid residues are derived from non-human CDRs and all or substantially all framework region (FR) amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. According to some embodiments, some FR residues in a humanized antibody are substituted with corresponding residues from a non-human antibody (e.g., the antibody from which the CDR residues are derived), e.g., to restore or improve antibody specificity or affinity.

The amino acid residues in the Fc domain can be substituted to be null, meaning the Fc domain does not bind Fc receptors or can bind with such low affinity and/or avidity as to not cause any Fc receptor signaling as a result of binding. The Fc domain can be null for binding to Fcγ receptors. Some example Fcγ receptors for which the Fc domain can be null for binding can be, but not limited to, FcγRI (CD64), FcγRIIA (CD32a), FcγRIIB (CD32b), FcγRIIIA (CD16a), FcγRIIIA (CD16a) F158 variant, FcγRIIIA (CD16a) V158 variant, or FcγRIIIB (CD16b). The Fc domain may have one or more, two or more, three or more, or four or more amino acid substitutions that decrease binding of the Fc domain to an Fc receptor.

According to some embodiments, conjugate comprise a humanized antibody having a mutated Fc domain that prevents FcγR-mediated internalization. According to some embodiments, the humanized antibody comprises a Fc null domain. According to certain embodiments, the Fc domain is null for binding to a Fcγ receptors.

As used herein, an “Fc null” refers to a domain that exhibits weak to no binding to one or more of the Fcγ receptors.

### Antibody-Drug Conjugates

The present invention provides a conjugate comprising the humanized antibody disclosed herein and a toxin.

According to some embodiments, the toxin is selected from the group consisting of microtubule inhibitor, DNA synthesis inhibitor, topoisomerase inhibitor, and RNA polymerase inhibitor. Each possibility represents a separate embodiment of the invention.

According to certain embodiments, the toxin is a microtubule-destroying drug. According to certain exemplary embodiments, the toxin is auristatin or a derivative thereof. According to certain embodiments, the auristatin derivative is monomethyl auristatin E (MMAE) or monomethyl auristatin F (MMAF).

According to some embodiments, the toxin is saponin.

According to some embodiments, the toxin is a maytansine derivative. According to certain embodiments, the maytansine derivative is DM4 or DM1.

According to some embodiments, the toxin is quinoline alkaloid. According to certain embodiments, the quinoline alkaloid is SN-38.

According to some embodiments, the toxin is a topoisomerase I inhibitor. According to some embodiments, the toxin is a derivative of camptothecin. According to certain exemplary embodiments, the toxin is Exatecan.

According to additional embodiments, the toxin is selected from the group consisting of MMAE, MMAF, Saporin, DM4, DM1, SN-38, Calicheamicin, DXd, exatecan, PBD, Duocarmycin, Sandramycin, alpha-Amanitin, Chaetocin, CYT997, Daunorubicin, 17-AAG, Agrochelin A, Doxorubicin, Methotrexate, Colchicine, Cordycepin, Epothilone B, Hygrolidin, Herboxidiene, Ferulenol, Curvulin, paclitaxel, Englerin A. Taltobulin, Triptolide, Cryptophycin, and Nemorubicin. Each possibility represents a separate embodiment of the invention.

According to some embodiments, the toxin is SN-38. According to some embodiments, the toxin is DM1. According to some embodiments, the toxin is DM4. According to some embodiments, the toxin is MMAE. According to some embodiments, the toxin is MMAF.

According to some embodiments, the antibody is directly linked to the toxin. According to other embodiments, the antibody and the toxin are linked through a linker. According to some embodiments, the humanized described herein is covalently linked to the toxin.

According to some embodiments, the linker is cleavable. According to additional embodiments, the linker is not cleavable.

According to some embodiments, the linker is cleaved in response to changes in pH or redox potential. According to some embodiments, the linker is cleaved when contacted with lysosomal enzymes.

According to some embodiments, the linker comprises a portion which is selected from the group consisting of 6-maleimidocaproyl (MC), maleimidopropionyl (MP), valine-citrulline (val-cit), alanine-phenylalanine (ala-phe), p-aminobenzyloxycarbonyl (PAB), N-succinimidyl 4-(2-pyridylthio) valerate (SPP), N-succinimidyl 4-(N-maleimidomethyl)-cyclohexane-1-carboxylate (SMCC), N-succinimidyl (4-iodo-acetyl)aminobenzoate (SLAB), 6-maleimidocaproyl-valine-citrulline-p-aminobenzyloxycarbonyl (MC-vc-PAB), Val-Cit-PABC, N-succinimidyl-4-(2-pyridyldithio) butanoate (SPDB), N-succinimidyl 3-(pyridin-2-yldithio)-propionate (SPDP), Phe-Lys (Fmoc)-PAB, Aloc-D-Ala-Phe-Lys (Aloc)-PAB-PNP, Boc-Phe-(Alloc) Lys-PAB-PNP, and perfluorophenyl 3-(pyridine-2-yldisulfanyl) propanoate. Each possibility represents a separate embodiment of the invention.

The present invention provides, according to another aspect, a pharmaceutical composition comprising the conjugate described herein and a pharmaceutically acceptable excipient, carrier, or diluent.

According to some embodiments, the pharmaceutical composition according to the invention is for use in treating cancer characterized by expression of PVR. According to other embodiments, the pharmaceutical composition according to the invention is for use in treating cancer characterized by overexpression of PVR. PVR expressing and overexpression related cancer types can be identified using known data bases such as The Cancer Genome Atlas (TCGA). According to certain embodiments, the cancer treatable with a composition according to the present invention is selected from the group consisting of glioblastoma multiforme (GBM), adrenocortical carcinoma (ACC), chromophobe renal cell carcinoma (KICH), liver hepatocellular carcinoma (LIHC), colon and rectal adenocarcinoma (COAD, READ), pancreatic ductal adenocarcinoma (PAAD), pheochromocytoma & paraganglioma (PCPG), papillary kidney carcinoma (KIRP), lung adenocarcinoma (LUAD), head and neck squamous cell carcinoma (HNSC), prostate adenocarcinoma (PRAD), uterine corpus endometrial carcinoma (UCEC), cervical cancer (CESC), cutaneous melanoma (SKCM), mesothelioma (MESO), urothelial bladder cancer (BLCA), clear cell kidney carcinoma (KIRC), lung squamous cell carcinoma (LUSC), uterine carcinosarcoma (UCS), sarcoma (SARC), ovarian serous cystadenocarcinoma (OV), papillary thyroid carcinoma (THCA), breast cancer (BRCA), lower grade glioma (LGG), and diffuse large B-cell lymphoma (DLBC). Each possibility represents a separate embodiment of the invention.

In seme embodiments, the ADCs provided herein are useful in treating hard-to-treat tumors. The term “hard-to-treat” as used herein refers to cancers associated with poor clinical outcomes, e.g., that their known therapies are not effective enough. Non limiting examples of ‘hard-to-treat’ tumors are lung, pancreatic, ovarian, colorectal and esophageal cancers, and brain tumors.

As used herein the term “individual,” “patient,” or “subject” refers to individuals diagnosed with, suspected of being afflicted with, or at-risk of developing at least one disease for which the described compositions and method are useful for treating. According to some embodiments the individual is a mammal. According to some embodiments, the mammal is a mouse, rat, rabbit, dog, cat, horse, cow, sheep, pig, goat, llama, alpaca, or yak. According to some embodiments, the individual is a human.

As used herein the term an “effective amount” refers to the amount of a therapeutic that causes a biological effect when administered to a mammal. Biological effects include, but are not limited to, reduced tumor growth, reduced tumor metastasis, or prolonged survival of an animal bearing a tumor. A “therapeutic amount” is the concentration of a drug calculated to exert a therapeutic effect. A therapeutic amount encompasses the range of dosages capable of inducing a therapeutic response in a population of individuals. The mammal can be a human individual. The human individual can be afflicted with or suspected or being afflicted with a tumor.

As used herein the term “combination” or “combination treatment” can refer either to concurrent administration of the articles to be combined or sequential administration of the articles to be combined. As described herein, when the combination refers to sequential administration of the articles, the articles can be administered in any temporal order.

As used herein “checkpoint inhibitor” refers a drug that inhibits a biological molecule (“checkpoint molecule”) produced by an organism that negatively regulates the anti-tumor/cancer activity of T cells in the organism. Checkpoint molecules include without limitation PD-1, PD-L-1, PD-L-2, CTLA4, TIM-3, LAG-3, VISTA, SIGLEC7, TIGIT, IDO, KIR, A2AR. B7-H3, B7H4, CEACAM1, and CD112R.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

The term “treatment” as used herein refers to both therapeutic treatment and prophylactic or preventative measures. Those in need of treatment include those already with the disorder as well as those in which the disorder is to be prevented.

The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

According to some embodiments, the ADCs described herein are used for treating hard-to-treat or resistant cancer.

The term “resistant cancer” as used herein refers to a cancer showing very low sensitivity to treatment with abundant anticancer drugs (e.g. chemotherapy) or radiation so that the cancer growth is not affected and symptoms thereof are not improved, relived, or alleviated by the drugs or radiation treatment, or cancer showing very low sensitivity to treatment with immune-modulatory agents, including but not limited to antibody-based molecules, immune cells, CAR cells, and cytokines. This term also refers to cases in which administration of certain therapies, such as radiation, is not possible. The term “resistant cancer” is used herein interchangeably with “hard-to-treat” cancer.

According to some embodiments, the ADC are used for treating subject with hard-to-treat cancer. According to certain embodiments, the subject has been already treated with chemotherapy and/or radiation.

According to some embodiments, the resistant cancer is selected from the group consisting of glioblastoma, pancreas cancer, colorectal cancer, liver cancer, lung cancer, skin cancer, ovarian cancer, esophageal cancer and endometrial cancer. According to some embodiments, the resistant cancer is glioblastoma (GBM).

According to some embodiments, the cancer is a resistant type of cancer. According to some embodiments, the cancer is characterized by expression of PVR. According to some embodiments, the cancer is characterized by high expression of PVR. According to some embodiments, the cancer is characterized by overexpression of PVR, the cancer cells comprise, in average, 2, 3, 4, 5, times more PVR molecules compared with a corresponding non-cancerous cell. According to some embodiments, the cancer is characterized by having >50% tumor proportion score (TPS) as evaluated by immunohistochemistry (IHC). According to certain embodiments, the cancer is characterized by having H score (histochemical score) of at least 1.

According to some embodiments, the method of treating cancer comprises administering the pharmaceutical composition as part of a treatment regimen comprising administration of at least one additional anti-cancer agent.

According to some embodiments, the anti-cancer agent is selected from the group consisting of an antimetabolite, a mitotic inhibitor, a taxane, a topoisomerase inhibitor, a topoisomerase II inhibitor, an asparaginase, an alkylating agent, an antitumor antibiotic, and combinations thereof. Each possibility represents a separate embodiment of the invention.

According to some embodiments, the antimetabolite is selected from the group consisting of cytarabine, fludarabine, fluorouracil, mercaptopurine, methotrexate, thioguanine, gemcitabine, and hydroxyurea. According to some embodiments, the mitotic inhibitor is selected from the group consisting of vincristine, vinblastine, and vinorelbine. According to some embodiments, the topoisomerase inhibitor is selected from the group consisting of topotecan and irinotecan. According to some embodiments, the alkylating agent is selected from the group consisting of busulfan, carmustine, lomustine, chlorambucil, cyclophosphamide, cisplatin, carboplatin, ifosfamide, mechlorethamine, melphalan, thiotepa, dacarbazine, and procarbazine. According to some embodiments, the antitumor antibiotic is selected from the group consisting of bleomycin, dactinomycin, daunorubicin, doxorubicin, idarubicin, mitomycin, mitoxantrone, and plicamycin. According to some embodiments, the topoisomerase II is selected from the group consisting of etoposide and teniposide. Each possibility represents a separate embodiment of the present invention.

The present invention provides, according to another aspect, a method of treating a cancer in an individual afflicted with a cancer comprising administering to the individual a therapeutically effective amount of the conjugate or the pharmaceutical composition, and an inhibitor of PD-1, PD-L1, CTLA-4 or CD112R signaling. In certain embodiments, the cancer comprises a solid tumor. In certain embodiments, the cancer is selected from the group consisting of lung cancer, colon cancer, glioblastoma, pancreatic cancer, breast cancer, bladder cancer, kidney cancer, head and neck cancer, ovarian cancer, cervical cancer, or prostate cancer. In certain embodiments, the inhibitor of PD-1 signaling is an antibody or fragment thereof that binds to PD-1. In certain embodiments, the antibody or fragment thereof that binds to PD-1 is Pembrolizumab, Nivolumab, AMP-514, Tislelizumab, Spartalizumab, or a PD-1 binding fragment thereof. In certain embodiments, the inhibitor of PD-1 signaling is an antibody that specifically binds PD-L-1 or PD-L-2. In certain embodiments, the antibody that specifically binds PD-L1 or PD-L2 comprises Durvalumab, Atezolizumab, Avelumab, BMS-936559, or FAZ053, or a PD-L1 or PD-L2 binding fragment thereof. In certain embodiments, the inhibitor of PD-1 signaling comprises an Fc-fusion protein that binds PD-1, PD-L1, or PD-L2. In certain embodiments, the Fc-fusion protein comprises AMP-224 or a PD-1 binding fragment thereof. In certain embodiments, the inhibitor of PD-1 signaling comprises a small molecule inhibitor of PD-1. PD-L1, or PD-L2. In certain embodiments, the small molecule inhibitor of PD-1, PD-L1, or PD-L2 signaling comprises on or more of: N-{2-[({2-methoxy-6-[(2-methyl[1,1′-biphenyl]-3-yl) methoxy]pyridin-3-yl}methyl)amino]ethyl}acetamide (BMS 202); (2-((3-cyanobenzyl)oxy)-4-((3-(2,3-dihydrobenzo[b][1,4]dioxin-6-yl)-2-methylbenzyl)oxy)-5-methylbenzyl)-D-serine hydrochloride; (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxylic acid; 3-(4,6-dichloro-1,3,5-triazin-2-yl)-1-phenylindole; 3-(4,6-dichloro-1,3,5-triazin-2-yl)-1-phenyl-1h-indole; L-α-Glutamine, N2,N6-bis(L-seryl-L-asparaginyl-L-threonyl-L-seryl-L-α-glutamyl-L-seryl-L- phenylalanyl)-L-lysyl-L-phenylalanyl-L-arginyl-L-valyl-L-threonyl-L-glutaminyl-L-leucyl-L-alanyl-L-prolyl-L-lysyl-L-alanyl-L-glutaminyl-L-isoleucyl-L-lysyl; (2S)-1-[[2,6-dimethoxy-4-[(2-methyl[1,1′-biphenyl]-3-yl) methoxy]phenyl]methyl]-2-piperidinecarboxylic acid; Glycinamide, N-(2-mercaptoacetyl)-L-phenylalanyl-N-methyl-L-alanyl-L-asparaginyl-L-prolyl-L-histidyl-L-leucyl-N-methylglycyl-L-tryptophyl-L-seryl-L-tryptophyl-N-methyl-L-norleucyl-N-methyl-L-norleucyl-L-arginyl-L- cysteinyl-, cyclic (1->14)-thioether; or a derivative or analog thereof.

Also described herein is a method of making composition for treating a cancer in an individual afflicted with cancer comprising admixing the conjugate and a pharmaceutically acceptable excipient, carrier, or diluent. In certain embodiments, the cancer comprises a solid tumor. In certain embodiments, the cancer is selected from the group consisting of glioblastoma, colon cancer, pancreatic cancer, breast cancer, bladder cancer, kidney cancer, head and neck cancer, ovarian cancer, cervical cancer, prostate cancer, and lung cancer.

According to some particular embodiments, the additional anti-cancer agent is selected from the group consisting of bevacizumab, carboplatin, cyclophosphamide, doxorubicin hydrochloride, gemcitabine hydrochloride, topotecan hydrochloride, thiotepa, and combinations thereof. Each possibility represents a separate embodiment of the present invention.

### EXAMPLES

Reference is now made to the following examples, which together with the above descriptions, illustrate the invention in a non-limiting fashion.

Generally, the nomenclature used herein, and the laboratory procedures utilized in the present invention include molecular, biochemical, microbiological, immunological, and recombinant DNA techniques. Such techniques are well known in the art. Other general references referring to well-known procedures are provided throughout this document for the convenience of the reader.

### Example 1. High Expression of PVR mRNA Correlates with Poor Survival Probability of Various Cancer Patients

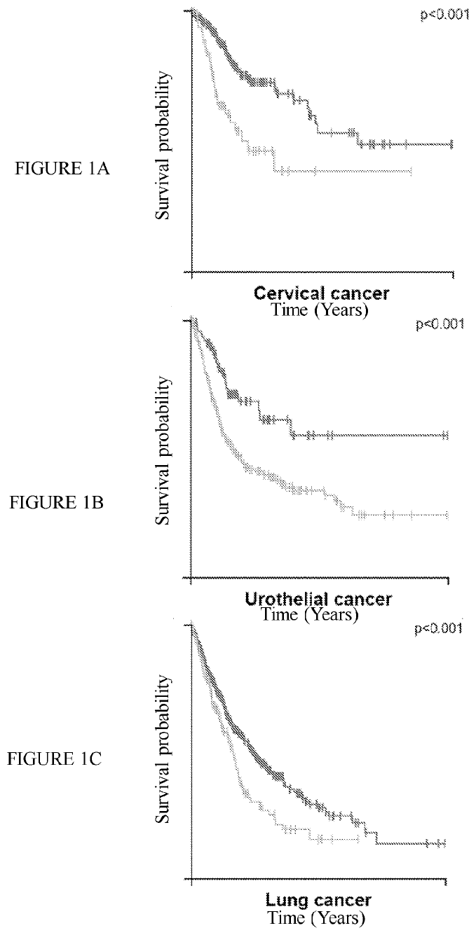

Based on the FPKM value of PVR, patients were classified into two expression groups and the correlation between PVR expression level and patient survival was examined. The prognosis of each group of patients was examined by Kaplan-Meier survival. Plots shown in FIG. 1 are for indications, in which PVR expression was defined as an unfavorable prognostic gene (meaning the risk of death in the high expression group is significantly p<0.001 higher than in the low expressing group), with significantly worse survival compared by log-rank tests.

### Example 2. PVR is Expressed on Majority of Solid Tumors

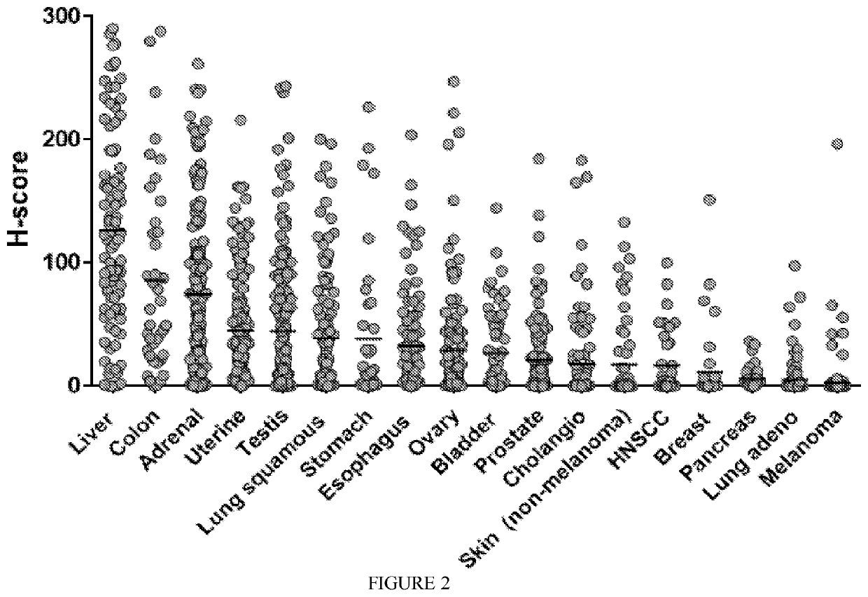

The expression levels of PVR in different human malignancies was evaluated. PVR expression was detected by standard immunohistochemistry procedures using the commercially available rabbit monoclonal antibody clone D3G7H, and cancer tissue microarrays. Staining was digitized and intensities were quantified to calculate H-scores within and across indications. FIG. 2 shows elevated expression levels of PVR in most indications analyzed, at varying frequencies. These data support the potential therapeutic benefit for multiple indications by targeting PVR via NTX1088-linked ADCs. The elevated expression of PVR was shown in liver cancer, colon cancer, adrenal cancer, uterine cancer, testicular cancer, squamous cell lung cancer, stomach cancer, esophagus cancer, ovary cancer, bladder cancer, prostate cancer, Cholangiocarcinoma, skin cancer, HNSCC cancer, breast cancer, pancreatic cancer, non-small cell lung cancer, and melanoma.

### Example 3. NTX1088 can Serve as an ADC Driver, and is Superior to Erbitux in Inducing EGFR⁺ PVR⁺ Cell Killing

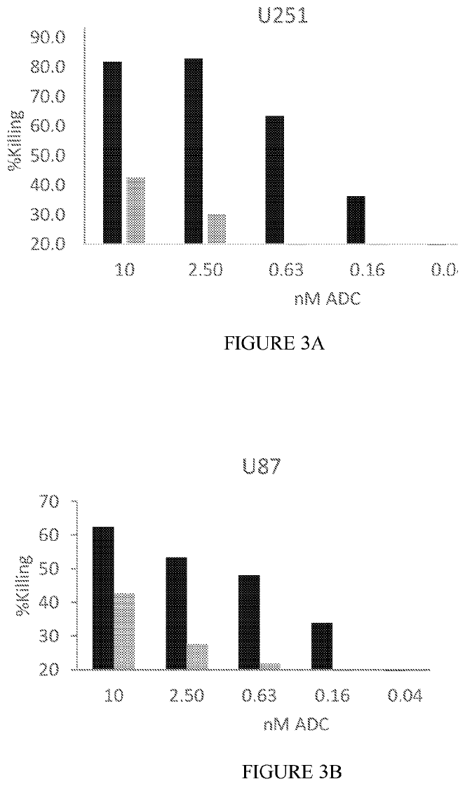

To assess the capacity of the mAbs to be used as ADCs, the streptavidin-saporin (ZAP, IT-27-250 (ATS)) was used. The mAbs indicated in FIG. 3 were biotinylated using a biotinylation kit (Ab207195, Abcam) at 1:1 ratio. FIG. 3 depicts the results of the initial screen which included NTX1088 and Erbitux targeting EGFR⁺ PVR⁺ GBM cell lines U251 and U87 as targets (FIGS. 3A and 3B, respectively). 2×10³ cells per well were plated and allowed to adhere over 4-6 hours period. The ADCs were added, and the cells were incubated with the ADCs for 96 hours after which tumor cell killing was evaluated using CellTiter-Glo® 2.0 Cell Viability Assay—(Promega G9242) standard protocol. NTX1088 exhibited significantly higher killing of both target cells compared to Erbitux.

### Example 4. NTX1088-ADC is Leading to Specific Killing of Targets Only when PVR is Expressed

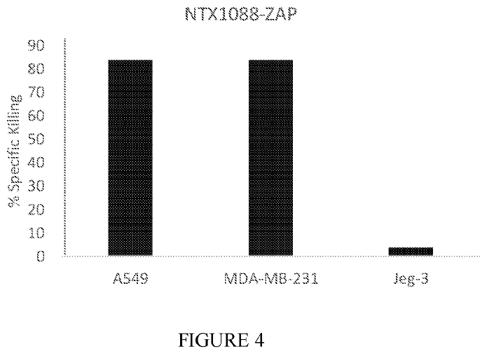

To assess the specificity of the NTX1088 based ADC, the streptavidin-saporin, described in FIG. 3, was used. FIG. 4 depicts the potency of a single dose (10 nM) against A549 (lung adenocarcinoma) and MDA-MB-231 (TNBC) target cells, which were confirmed to express PVR. Jeg-3 (Choriocarcinoma) cells, which do not express PVR, were used to evaluate the specificity of the ADC. 2×10³ cells per well were plated and allowed to adhere over 4-6 hours period. The ADCs were added, and the cells were incubated with the ADCs for 96 hours. After 96 hours, the assay was harvested and tumor cell killing was evaluated using CellTiter-Glo® 2.0 Cell Viability Assay-(Promega G9242) standard protocol. As shown in FIG. 4, specific killing of targets positive for PVR expression was more than 20-folds higher compared to the killing of the PVR-negative target cells, supporting highly specific activity, and indicating prospective safety, of the NTX1088-based ADC.

### Example 5. NTX1088-Based ADC Evaluation by a Matrix of Linker Payloads

The presented linker-payload combinations were chosen according to the desired release mechanism in combination with different payloads. These ADCs were generated according to standard protocols by Abzena LTD. Briefly, the mAbs were reduced and incubated with an excess of the linker-payload to obtain the desired drug antibody ratio (DAR) of 4 for all the linker-payloads except for SN-38, which had a target DAR of 8. The final product was then purified, and the DAR was established by LC/MS analysis. A summary of the ADCs properties is presented in Table 1.

TABLE 1
A summary of the linker-payload combinations, release
mechanisms, and the relevant drug-to-antibody
ratios (DARs) for the NTX1088-based ADCs.

| | | | | Average |
|---|---|---|---|---|
| | | | Release | DAR (LC- |
| ADC name | Linker | Payload | mechanism | MS) |
| NTX1088- | MC-VC-PAB | MMAE | Proteolytic | 3.8 |
| MMAE | | | cleavage | |
| NTX1088- | MC | MMAF | Degradation | 4.0 |
| MMAF | | | | |
| NTX1088-DM1 | SMCC | DM1 | Degradation | 3.8 |
| NTX1088-DM4 | SPDB | DM4 | Redox | 3.8 |
| NTX1088- | Lys-PAB-CO | SN38 | pH | 8.4 |
| SN38 | | | | |

### Example 6. NTX1088 Based ADC Potency Evaluation of a Matrix of Linker Payloads

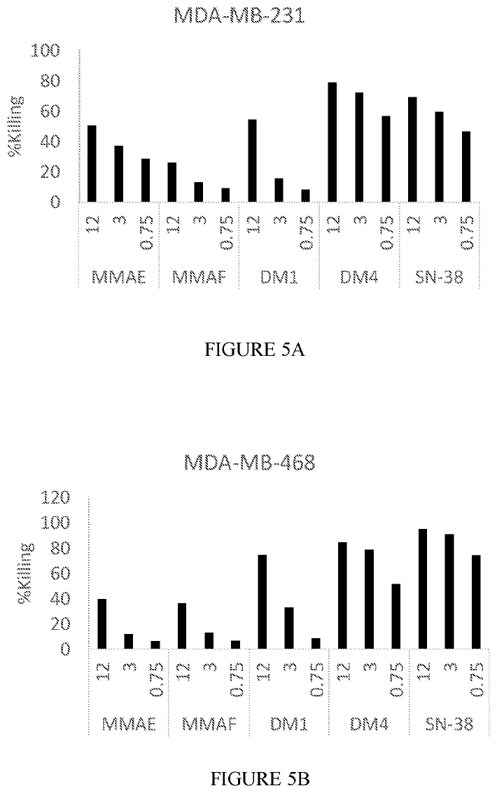

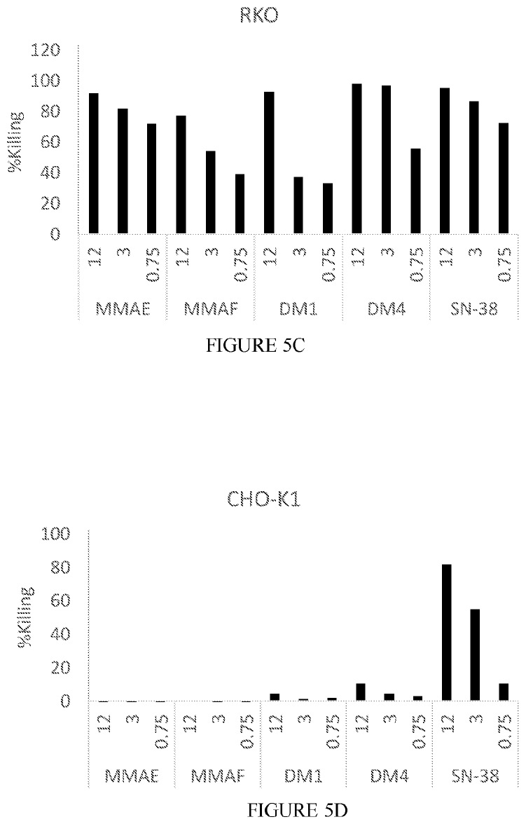

Selected tumor cell lines representing various solid tumors were used to assess the in vitro potency of the various linker-payload combinations described in Table 1. The indicated target cells were plated at 2×10³ cells per well and allowed to adhere over 4-6 hours period. The ADCs were added at concentrations of 12-0.75 ug/ml using 4-fold dilutions, and the cells were incubated with the ADCs for additional 72 hours. Then, tumor cell killing was evaluated using CellTiter-Glo® 2.0 Cell Viability Assay-(Promega G9242) following a standard protocol. Robust killing of MDA-MB-231 cells (triple negative breast cancer), MDA-MB-468 cells (triple negative breast cancer) and RKO cells (colorectal adenocarcinoma) is depicted in FIGS. 5A-5C, respectively. CHO cells, which do not express human PVR, were not killed by most linker payload combinations, except the carbonate bound SN-38, which is released to the culture media in a non-specific manner. Cytotoxicity was significant and specific for all payload types, with DM4 having the most potent effect. Of note, at 0.75 ug/ml, SN-38 led to extremely minor killing of the CHO cells, while exceeding 70% killing for RKO and MDA-MB-468 cells, suggesting that there is also a significant PVR-specific killing component to this ADC.

While there was clear impact of the target type on the activity, the most promising candidates were NTX1088-MMAE, NTX1088-DM4 and NTX1088-SN38.

### Example 7. NTX1088 Based ADCs are Potent Against Cell Lines Representative of Hard-to-Treat Solid Cancers

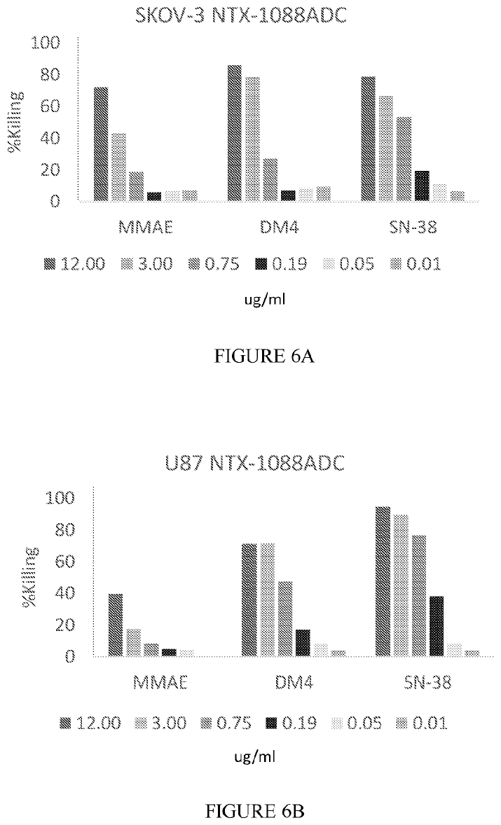

Selected tumor cell lines, representing hard-to-treat solid tumors, were used to assess potency of the lead selected linker-payload NTX1088-ADC in-vitro. The killing assay was performed as described in FIG. 5 for ADC range of 12-0.01 ug/ml using 4-fold dilutions. Robust killing of SKOV-3 cells (ovarian cancer), and U87 cells (GBM) are shown in FIGS. 6A-6B, respectively. Significant killing p<0.01 is seen at all doses up to 0.75 ug/ml for all linker-payloads. Surprisingly, the killing of U87 cells is significantly lower for both NTX1088-DM4 and NTX1088-MMAE, which did not reach EC-50 at the highest ADC concentration, suggesting this is a model for an extremely resistant cancer.

### Example 8. Selected NTX1088 Based ADCs Lead to Tumor Regression in an Aggressive GBM In Vivo Model

Nude female mice were injected SC with 5×10⁶ U87 cells in 1:1 Matrigel. Once tumors reached an average volume of 160 mm³ mice were randomized into four groups (n=7 per group) and treated, in a blinded manner, by i.v. injection of either PBS (vehicle), NTX1088-MMAE, NTX1088-DM4, or NTX1088-SN38. A dose of 5 mg/kg was administered every 4 days for 3 consecutive doses, followed by a single 5 mg/kg dose on day 25 post last dose.

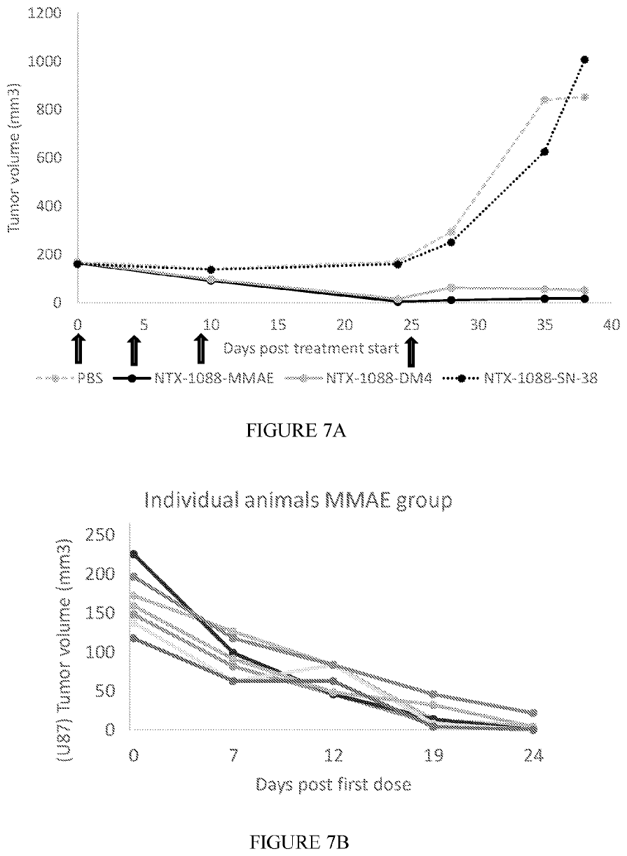

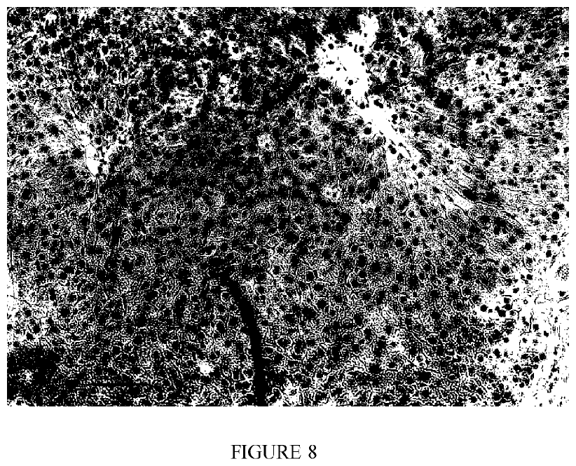

As shown in FIG. 7, when compared to the control group, there was no effect on tumor growth in the group treated by NTX1088-SN38, despite the high potency of this ADC toward the U87 target seen in vitro (FIG. 6B). NTX1088-DM4 resulted in near complete tumor regression, in line with the high potency of this linker payload seen in vitro. Finally, in a manner unpredicted by the in vitro results, the NTX1088-MMAE was the most potent ADC, resulting in complete tumor regression. These results are in sharp contrast to previously attempts to use other anti-PVR ADCs against highly resistant tumor cells, such as U87. For example, in WO 2019/102456, the described anti-PVR ADC had no impact on tumor growth of U87 cells (FIG. 6B of WO 2019/102456). Unexpectedly, the tested NTX1088-DM4 and NTX1088-MMAE (but not NTX1088-SN38) were able to exert tumor regression. These results confirm that NTX1088-ADCs, are novel and nonobvious therapeutic agents.

Combined these findings support the clinical development of a novel class of drugs, NTX1088-based ADCs for the treatment of solid tumors.

These in vivo findings are unexpected given the much different activity profiles seen at the in vitro assays (FIGS. 5 and 6).

### Example 9. NTX1088 ADC has a Clean Safety Profile In Vivo when Tested in Human-PVR Expressing Mice

TgPVR21 mice, a strain expressing full length human PVR (doi: 10.1073/pnas.88.3.951) are an accepted mouse model for testing the virulence of poliovirus vaccines. We used these animals to evaluate the safety of the most potent NTX1088-ADC. Based on above results, NTX1088-MMAE was selected. TgPVR21 mice are reported to express human PVR at multiple tissues. For us, the liver was considered to be the most relevant target organ, since the human liver express the highest level of PVR out of all normal tissues (The Human Protein Atlas (proteinatlas.org)). To test the expression of human PVR on the mouse liver cells, the IHC described above was utilized (Example 3), and the commercial anti-PVR antibody clone D37GH which is not cross-reactive to mouse PVR was used. FIG. 8 shows representative histology data from a liver of a female PVRTg21 mouse. The tested samples exhibit robust membranal expression of PVR across the entire tissue. Given the nature of the MMAE toxin, its toxicity should be evident upon acute exposure. Thus, next, 3 PVRTg21 female mice per group (age 13 weeks) were injected iv with a single dose of PBS or 7.5 mg/kg or 10 mg/kg of NTX1088-MMAE. Toxicity was assessed by collecting blood samples from the treated mice on days 1 and 10 post dosing (Table 2 and Table 3, respectively). The levels of liver enzymes were measured and normalized to the animals treated with PBS. No meaningful changes in liver enzymes were seen at any time point at any of the treated groups. No weight loss was seen during this period as well (data not shown). These findings suggest that NTX1088 is safe and well tolerated even when PVR is broadly expressed on normal tissues. The safety window obtained (no toxic effects) is the result of combining the antibody with a specific payload preferentially targeting dividing cells. This outcome is highly unexpected, especially given the robust anti-tumor effect seen using a fraction of this dose (example 8). Cumulatively, it is now shown that NTX1088 can be used as a safe and potent ADC for the treatment of cancers positive for the expression of PVR.

TABLE 2
Blood toxicity of PVRTg21 female mice injected iv with
a single dose of PBS or 7.5 mg/kg or 10 mg/kg of NTX1088-
MMAE. Blood samples from day 1 post dosing.

| | Fold Change from Vehicle | | | |
|---|---|---|---|---|
| | | Alkaline | | |
| | T. Bil¹ | Phosphatase | | |
| Day −1 | (mg/dl) | (IU/L) | SGOT²(IU/L) | SGPT³(IU/L) |
| Vehicle (PBS) | 1.00 | 1.00 | 1.00 | 1.00 |
| 1088-MMAE | 1.13 | 0.86 | 1.06 | 1.13 |
| 7.5 mg/kg | | | | |
| 1088-MMAE | 0.88 | 0.87 | 1.10 | 0.84 |
| 10 mg/kg | | | | |

¹Total bilirubin.
²Serum glutamic-pyruvic transaminase.
³Serum glutamic-oxaloacetic transaminase.

TABLE 3
Blood toxicity of PVRTg21 female mice injected iv with
a single dose of PBS or 7.5 mg/kg or 10 mg/kg of NTX1088-
MMAE. Blood samples from day 10 post dosing.

| | Fold Change from Vehicle | | | |
|---|---|---|---|---|
| | | Alkaline | | |
| | T. Bil¹ | Phosphatase | | |
| Day −10 | (mg/dl) | (IU/L) | SGOT²(IU/L) | SGPT³(IU/L) |
| Vehicle | 1.00 | 1.00 | 1.00 | 1.00 |
| (PBS) | | | | |
| 1088- | 0.77 | 1.04 | 0.92 | 0.93 |
| MMAE | | | | |
| 7.5 mg/kg | | | | |
| 1088- | 0.92 | 1.17 | 0.97 | 0.78 |
| MMAE | | | | |
| 10 mg/kg | | | | |

¹Total bilirubin.
²Serum glutamic-pyruvic transaminase.
³Serum glutamic-oxaloacetic transaminase.

### Example 9. NTX1088-Based ADCs Show High Treatment Efficacy in NSCLC Model H322M

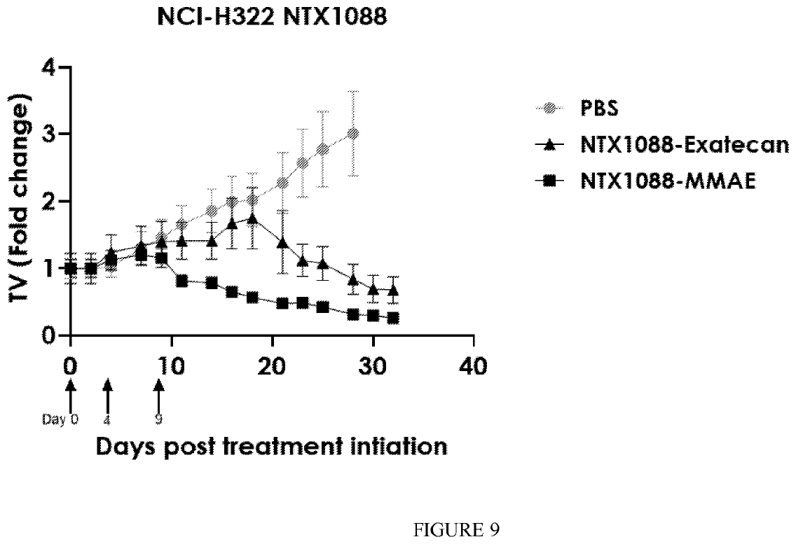

Nude female mice were injected SC with 5×10⁶ H322M cells in 1:1 Matrigel. Once tumors exceeded 130 mm³, mice were randomized into three groups (n=6 per group) and treated, in a blinded manner, by i.v. injections of either PBS, 5 mg/kg NTX1088-MMAE or 10 mg/kg NTX1088-Exatecan on days 0, 4 and 9. As shown in FIG. 9, both ADC treated groups exhibited initial tumor growth, followed by a robust tumor inhibition compared to the control (PBS) group, followed by tumor regression as compared to tumor volume at treatment initiation. These results demonstrate that NTX1088-based ADCs can have a robust therapeutic utility when conjugated to tubulin or Topol targeting agents. The kinetics of the effect were different between the two NTX1088 ADCs, demonstrating different impact of specific Ab-drug combinations which may not be predicted.

### Sequences

TABLE 4
Antibody sequences

| SEQ ID | | |
|---|---|---|
| NO: | Sequence | Chain |
| 1 | QVQLVQSGAEVKKPGASVKVSCKATGYTFSNYWIEWVRQAPGQG | NTX1088 |
| | LEWIGEIFPGSGRINFNEKFKGRVTFTADTSISTTYMELSRLRSDDTA | Heavy chain |
| | VYYCARTKIYGNSFDYWGQGTLVTVSS | (VH4) |
| 2 | DIQMTQSPSSLSASVGDRVTITCKASQDVGTAVVWYQQKPGKAPKL | NTX1088 |
| | LIYWASSRHEGVPDRFSGSGSGTDFTLTISSLQPEDFADYFCQQYSR | Light chain |
| | YPLTFGQGTKLEIK | (VK2) |
| 3 | NYWIE | HCCDR1 |
| 4 | EIFPGSGRINFNEKFKG | HCCDR2 |
| 5 | TKIYGNSFDY | HCCDR3 |
| 6 | KASQDVGTAVV | LCCDR1 |
| 7 | WASSRHE | LCCDR2 |
| 8 | QQYSRYPLT | LCCDR3 |
| 9 | QVQLVQSGAE(L/V)KKPGASVK(I/V)SCKATGYTFSNYWIEW(I/V) | Heavy chain |
| | (K/R)QAPGQGLEW(I/M)GEIFPGSGRINFNEKFKGR(A/V)TFTADTSI | formula |
| | (D/S)T(T/A)YM(Q/E)LS(S/R)L(T/R)SDD(S/T)AVYYCARTKIYGNSFDYWG | |
| | QGT(T/L)VTVSS | |
| 10 | DI(M/Q)MTQSPS(F/S)LSASVGDRVTITC(K/R)ASQDVGTAV(V/A)WY | Light chain |
| | QQKPGKAPK(L/S)LIYWASSRHEGVP(D/S)RF(T/S)GSGSGTDFTLTIS | formula |
| | SLQ(S/P)EDFA(D/T)YFCQQYSRYPLTFGQGT KLEIK | |
| 11 | QVQLVQSGAELKKPGASVKISCKATGYTFSNYWIEWIKQAPGQGLE | Heavy chain |
| | WIGEIFPGSGRINFNEKFKGRATFTADTSIDTTYMQLSSLTSDDSAVY | VH1 |
| | YCARTKIYGNSFDYWGQGTTVTVSS | |
| 12 | QVQLVQSGAEVKKPGASVKISCKATGYTFSNYWIEWIKQAPGQGLE | Heavy chain |
| | WIGEIFPGSGRINFNEKFKGRATFTADTSIDTTYMELSRLRSDDTAVY | VH2 |
| | YCARTKIYGNSFDYWGQGTLVTVSS | |
| 13 | QVQLVQSGAEVKKPGASVKVSCKATGYTFSNYWIEWIKQAPGQGL | Heavy chain |
| | EWIGEIFPGSGRINFNEKFKGRVTFTADTSISTTYMELSRLRSDDTAV | VH3 |
| | YYCARTKIYGNSFDYWGQGTLVTVSS | |
| 14 | QVQLVQSGAEVKKPGASVKVSCKATGYTFSNYWIEWVRQAPGQG | Heavy chain |
| | LEWMGEIFPGSGRINFNEKFKGRVTFTADTSISTAYMELSRLRSDDT | VH5 |
| | AVYYCARTKIYGNSFDYWGQGTLVTVSS | |
| 15 | DIMMTQSPSFLSASVGDRVTITCKASQDVGTAVVWYQQKPGKAPK | Light chain |
| | LLIYWASSRHEGVPDRFTGSGSGTDFTLTISSLQSEDFADYFCQQYS | VK1 |
| | RYPLTFGQGTKLEIK | |
| 16 | DIQMTQSPSSLSASVGDRVTITCRASQDVGTAVVWYQQKPGKAPKL | Light chain |
| | LIYWASSRHEGVPSRFSGSGSGTDFTLTISSLQPEDFATYFCQQYSRY | VK3 |
| | PLTFGQGTKLEIK | |
| 17 | DIQMTQSPSSLSASVGDRVTITCRASQDVGTAVAWYQQKPGKAPKS | Light chain |
| | LIYWASSRHEGVPSRFSGSGSGTDFTLTISSLQPEDFATYFCQQYSRY | VK4 |
| | PLTFGQGTKLEIK | |
| 18 | QVQLVQSGAEVKKPGASVKVSCKATGYTFSNYWIEWVRQAPGQGLEWIGEIFPGSGRINFNE | Heavy chain |
| | KFKGRVTFTADTSISTTYMELSRLRSDDTAVYYCARTKIYGNSFDYWGQGTLVTVSSASTKG | full length |
| | PSVFPLAPCSRSTSESTAALGCLVKDYFPEPVTVSWNSGALTSGVHTEPAVLQSSGLYSLSS | hIgG4 |
| | VVTVPSSSLGTKTYTCNVDHKPSNTKVDKRVESKYGPPCPPCPAPEELGGPSVFLFPPKPKD | (S241P)(VH4) |
| | TLMISRTPEVTCVVVDVSQEDPEVQFNWYVDGVEVHNAKTKPREEQFNSTYRVVSVLTVLHQ | |
| | DWLNGKEYKCKVSNKGLPSSIEKTISKAKGQPREPQVYTLPPSQEEMTKNQVSLTCLVKGFY | |
| | PSDIAVEWESNGQPENNYKTTPPVLDSDGSFFLYSRLTVDKSRWQEGNVFSCSVMHEALHNH | |
| | YTQKSLSLSLGK | |
| 19 | DIQMTQSPSSLSASVGDRVTITCKASQDVGTAVVWYQQKPGKAPKL | Light chain |
| | LIYWASSRHEGVPDRFSGSGSGTDFTLTISSLQPEDFADYFCQQYSR | full length |
| | YPLTFGQGTKLEIKRTVAAPSVFIFPPSDEQLKSGTASVVCLLNNFYP | (Vk2) |
| | REAKVQWKVDNALQSGNSQESVTEQDSKDSTYSLSSTLTLSKADYE | |
| | KHKVYACEVTHQGLSSPVTKSFNRGEC | |

1-39. (canceled)

40. An antibody-drug conjugate (ADC) comprising a humanized anti-PVR antibody, or antigen binding portion thereof, conjugated to a toxin, the antibody or antigen binding portion thereof comprises a heavy chain and a light chain, wherein the heavy chain comprises a variable region having an amino acid sequence at least about 90% identical to SEQ ID NO: 1, and wherein the light chain comprises a variable region having an amino acid sequence at least about 90% identical to SEQ ID NO: 2.

41. The antibody-drug conjugate of claim 40, wherein the heavy-chain variable region comprises the CDR sequences set forth in SEQ ID NO: 3 (NYWIE), SEQ ID NO: 4 (EIFPGSGRINFNEKFKG) and SEQ ID NO: 5 (TKIYGNSFDY), and the light-chain variable region comprises the CDR sequences set forth in SEQ ID NO: 6 (KASQDVGTAVV), SEQ ID NO: 7 (WASSRHE) and SEQ ID NO: 8 (QQYSRYPLT).

42. The antibody-drug conjugate of claim 40, wherein the humanized antibody comprising a heavy chain comprising a variable region having an amino acid sequence at least about 95% identical to SEQ ID NO: 1, and wherein the humanized antibody comprising a light chain comprising a variable region having an amino acid sequence at least about 95% identical to SEQ ID NO: 2.

43. The antibody-drug conjugate of claim 40, wherein the humanized antibody comprises a heavy chain comprising a variable region having an amino acid sequence set forth in SEQ ID NO: 1 and a light chain comprising a variable region having an amino acid sequence set forth in SEQ ID NO: 2.

44. The antibody-drug conjugate of claim 40, wherein the toxin is selected from the group consisting of microtubule inhibitor, DNA synthesis inhibitor, topoisomerase inhibitor and RNA polymerase inhibitor.

45. The antibody-drug conjugate of claim 40, wherein the toxin is selected from the group consisting of auristatin or a derivative thereof, a maytansine derivative, and quinoline alkaloid.

46. The antibody-drug conjugate of claim 40, wherein the toxin is selected from the group consisting of Exatecan, DM4, MMAE and SN-38.

47. The antibody-drug conjugate of claim 40, wherein the conjugate comprises the toxin Exatecan.

48. The antibody-drug conjugate of claim 40, wherein the antibody and the toxin are linked through a linker.

49. The antibody-drug conjugate of claim 48, wherein the linker is cleavable.

50. The antibody-drug conjugate of claim 49, wherein the linker comprises a moiety selected from the group consisting of Valine-Alanine (VA), Maleimidocaproyl (MC), Maleimidocaproyl-Valine-Citrulline-p-amino-benzyloxycarbonyl (MC-VC-PAB), Maleimidomethyl cyclohexane-1-carboxylate (SMCC), N-succinimidyl-4-(2-pyridyldithio) butanoate (SPDB), and Lys-PAB-CO (Lysine-p-aminobenzyl-C═O).

51. The antibody-drug conjugate of claim 40, wherein the conjugate comprises (i) a toxin Exatecan and a linker Valine-Alanine (VA); (ii) a toxin MMAE and a linker MC-VC-PAB (denoted herein NTX1088-MMAE);
(iii) a toxin MMAF and a linker MC (denoted herein NTX1088-MMAF);
(iv) a toxin DM1 and a linker SMCC (denoted herein NTX1088-DM1);
(v) a toxin DM4 and a linker SPDB (denoted herein NTX1088-DM4); or
(vi) a toxin SN38 and a linker Lys-PAB-CO (denoted herein NTX1088-SN38).

52. The antibody-drug conjugate of claim 40 wherein the antibody comprises a heavy chain variable region having a sequence set forth in SEQ ID NO: 1, and a light chain variable region having a sequence set forth in SEQ ID NO: 2, wherein the linker comprises Valine-Alanine (VA), and wherein the toxin is Exatecan.

53. A pharmaceutical composition comprising the antibody-drug conjugate according to claim 40 and a pharmaceutically acceptable excipient, carrier, or diluent.

54. A method of treating a cancer in an individual in need of such treatment, the method comprising administering to the individual a therapeutically effective amount of the pharmaceutical composition of claim 53.

55. The method of claim 54, wherein the cancer is resistant to chemotherapy or radiation.

56. The method of claim 54, wherein the cancer is a hard-to-treat cancer.

57. The method of claim 54, wherein the cancer is selected from the group consisting of prostate cancer, ovarian cancer, colorectal cancer, breast cancer, pancreatic cancer, liver cancer, lung cancer, glioblastoma, adrenal cancer, uterine cancer, testis cancer, and head and neck cancer.

58. A conjugate comprising a humanized anti-PVR antibody, or antigen binding portion thereof, conjugated to a detectable moiety, a radioactive moiety, or labeling tag, the antibody or antigen binding portion thereof comprises a heavy chain and a light chain, wherein the heavy chain comprises a variable region having an amino acid sequence at least about 90% identical to SEQ ID NO: 1, and wherein the light chain comprises a variable region having an amino acid sequence at least about 90% identical to SEQ ID NO: 2.

59. A method of diagnosing or prognosing cancer in a subject, the method comprises determining the expression level of PVR in a biological sample of said subject using at least one conjugate according to claim 58.
